(19) United States
(12) Patent Application Publication
(10) Pub. No.: US 2010/0204296 A1
(43) Pub. Date: Aug. 12, 2010

(54) Novel Polymorphs of Darifenacin Free Base and its Hydrobromide Salt

(75) Inventors: Eswara Rao Kodali, Andhra Pradesh (IN); Sreekanth Medikonduri, Andhra Pradesh (IN); Praveen Kumar Neela, Andhra Pradesh (IN); Nitin Sharadchandra Pradhan, Maharashtra (IN); Jon Valgeirsson, Hafnarfjordur (IS)

Correspondence Address: CANTOR COLBURN, LLP, 20 Church Street, 22nd Floor, Hartford, CT, 06103, US

(73) Assignee: ACTAVIS GROUP PTC EHF, Hafnarfjordur (IS)

(21) Appl. No.: 12/663,558

(22) PCT Filed: Jun. 9, 2008
(86) PCT No.: PCT/IB08/02571

§ 371 Date: Apr. 16, 2010

(30) Foreign Application Priority Data
Jun. 8, 2007 (IN) 1186/CHE/2007
Jul. 13, 2007 (IN) 1512/CHE/2007

(51) Int. Cl. A61K 31/4025; C07D 405/02

(52) U.S. Cl. 514/422; 548/525

(57) ABSTRACT

The present invention provides a novel and stable amorphous form of darifenacin free base, process for preparation, pharmaceutical compositions, and method of treating thereof. The present invention further provides a novel and stable polymorphic form of darifenacin hydrobromide, process for preparation, pharmaceutical compositions, and method of treating thereof.

### CROSS REFERENCE TO RELATED APPLICATION

This application claims the benefit of priority to Indian provisional application Nos. 1186/CHE/2007, filed on Jun. 8, 2007, and 1512/CHE/2007, filed on Jul. 13, 2007, which are incorporated herein by reference.

### FIELD OF THE INVENTION

The present invention provides a novel amorphous form of darifenacin free base, process for preparation, pharmaceutical compositions, and method of treating thereof. The present invention further provides a novel polymorphic form of darifenacin hydrobromide, process for preparation, pharmaceutical compositions, and method of treating thereof.

### BACKGROUND OF THE INVENTION

Darifenacin, also known as (S)-2-[1-[2-(2,3-dihydrobenzofuran-5-yl)ethyl]-3-pyrrolidinyl]-2,2-diphenylacetamide, is represented by the following structural formula:

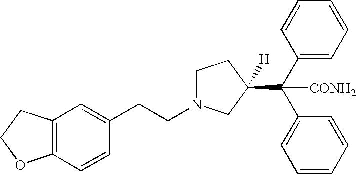

Darifenacin is a competitive muscarinic receptor antagonist. Muscarinic receptors play an important role in several major cholinergically mediated functions, including contractions of the urinary bladder smooth muscle and stimulation of salivary secretion. Darifenacin is administered as the hydrobromide salt, and is marketed under the trade names ENABLEX® and EMSELEX® by Novartis. Darifenacin hydrobromide and three routes for its preparation are disclosed in U.S. Pat. No. 5,096,890.

U.S. Patent Application No. 20050245597 discloses stable solid hydrate form of darifenacin free base (characterized by an X-ray powder diffraction pattern having peaks expressed as 2-theta at about 8.39, 10.519, 13.272, 13.693, 15.908, 16.289, 16.855, 19.637, 21.135, 21.55, 21.722, 23.006, and 26.284 degrees), pharmaceutical composition, method of use and process for the preparation thereof. Amorphous darifenacin base is not disclosed in the prior art.

IP.com Electronic Publication No. IPCOM000137408D, published on Jun. 19, 2006 (herein after referred to as the ‘IP.com publication’) discloses two crystalline forms of darifenacin hydrobromide (see FIG. 13 and FIG. 17), characterizes them by powder X-ray diffraction (P-XRD), Infra red (IR) spectroscopy and Differential Scanning Calorimetry (DSC). According the IP.com publication, the first crystalline form (Emselex 15 mg tablet) is characterized by an X-ray powder diffraction pattern having peaks expressed as 2-theta at about 8.2, 9.1, 11.5, 12.6, 13.2, 14.4, 16.3, 17.1, 17.8, 18.3, 18.9, 19.2, 20.3, 20.8, 22.1, 24.1, 24.7, 25.2, 25.6, 26.5, 26.6, 27.1, 28.6, 28.9, 30.3, 30.5±0.2 degrees; the second crystalline form (precipitated from acetone, methanol, acetonitrile or methylene dichloride) is characterized by an X-ray powder diffraction pattern having peaks expressed as 2-theta at about 8.2, 9.1, 11.5, 12.5, 14.4, 16.7, 17.1, 17.3, 17.8, 18.3, 18.8, 19.2, 20.1, 20.3, 20.8, 22.1, 23.7, 24.1, 24.7, 25.2, 25.9, 26.2, 26.8, 27.4, 27.6, 28.1, 28.9, 30.0±0.2 degrees.

Polymorphism is defined as “the ability of a substance to exist as two or more crystalline phases that have different arrangement and/or conformations of the molecule in the crystal lattice. Thus, in the strict sense, polymorphs are different crystalline forms of the same pure substance in which the molecules have different arrangements and/or configurations of the molecules”. Different polymorphs may differ in their physical properties such as melting point, solubility, X-ray diffraction patterns, etc. Although those differences disappear once the compound is dissolved, they can appreciably influence pharmaceutically relevant properties of the solid form, such as handling properties, dissolution rate and stability. Such properties can significantly influence the processing, shelf life, and commercial acceptance of a polymorph. It is therefore important to investigate all solid forms of a drug, including all polymorphic forms, and to determine the stability, dissolution and flow properties of each polymorphic form. Polymorphic forms of a compound can be distinguished in the laboratory by analytical methods such as X-ray diffraction (XRD), Differential Scanning Calorimetry (DSC) and infrared spectrometry (IR).

Solvent medium and mode of isolation play very important role in obtaining a polymorphic form over the other.

It has been disclosed in the art that the amorphous forms in a number of drugs exhibit superior dissolution characteristics and in some cases different bioavailability patterns compared to crystalline forms [Konne T., Chem. Pharm. Bull., 38, 2003 (1990)]. For some therapeutic indications one bioavailability pattern may be favored over another. An amorphous form of cefuroxime axetil is good example for exhibiting higher bioavailability than the crystalline forms.

The discovery of new polymorphic forms of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic.

Accordingly, there remains a need in the art for substantially pure amorphous darifenacin base. Since the amorphous darifenacin base obtained with high purity, the said darifenacin base can be used to obtain pharmaceutically acceptable salts of darifenacin in high purity. It has been found that purification of impure darifenacin base is practically advantageous when compared with the purification of a salt of it.

There is also a need in the art for novel and stable polymorphic form of darifenacin hydrobromide salt.

### SUMMARY OF THE INVENTION

We have now surprisingly and unexpectedly discovered novel polymorphic forms of darifenacin free base and its hydrobromide salt with adequate stability and good dissolution properties.

In one aspect, the present invention provides a novel and stable amorphous form of darifenacin free base and use thereof for the preparation of darifenacin hydrobromide.

In another aspect, the present invention further encompasses a process for preparing the highly pure and stable amorphous form of darifenacin free base.

In another aspect, the present invention provides a novel and stable polymorphic form of darifenacin hydrobromide, designated as darifenacin hydrobromide polymorphic form A1, characterized by an X-ray powder diffraction pattern having peaks expressed as 2-theta angle positions at about 23.44, 27.12, and 36.68±0.2 degrees 2-theta.

In another aspect, the present invention further encompasses a process for preparing the highly pure and stable polymorphic form A1 of darifenacin hydrobromide.

In another aspect, the present invention provides a pharmaceutical composition comprising amorphous form of darifenacin free base of the present invention and one or more pharmaceutically acceptable excipients.

In still another aspect, the present invention provides a pharmaceutical composition comprising amorphous form of darifenacin free base made by the process of the present invention, and one or more pharmaceutically acceptable excipients.

In still further aspect, the present invention further encompasses a process for preparing a pharmaceutical formulation comprising combining amorphous form of darifenacin free base with one or more pharmaceutically acceptable excipients.

In another aspect, the present invention provides a pharmaceutical composition comprising darifenacin hydrobromide polymorphic form A1 of the present invention and one or more pharmaceutically acceptable excipients.

In still another aspect, the present invention provides a pharmaceutical composition comprising darifenacin hydrobromide polymorphic form A1 made by the process of the present invention, and one or more pharmaceutically acceptable excipients.

In still further aspect, the present invention further encompasses a process for preparing a pharmaceutical formulation comprising combining darifenacin hydrobromide polymorphic form A1 with one or more pharmaceutically acceptable excipients.

Unless otherwise indicated, the following definitions are set forth to illustrate and define the meaning and scope of the various terms used to describe the invention herein.

The term “polymorphic form” refers to a crystal modification that can be characterized by analytical methods such as X-ray powder diffraction, IR-spectroscopy, differential scanning calorimetry (DSC) or by its melting point.

The term “amorphous” means a solid without long-range crystalline order. Amorphous form of darifenacin free base in accordance with the present invention preferably contains less than about 10% crystalline forms of darifenacin free base, more preferably less than 5% crystalline forms of darifenacin free base, and still more preferably is essentially free of crystalline forms of darifenacin free base. “Essentially free of crystalline forms of darifenacin free base” means that no crystalline polymorph forms of darifenacin free base can be detected within the limits of a powder X-ray di ffractometer.

The term “pharmaceutically acceptable” means that which is useful in preparing a pharmaceutical composition that is generally non-toxic and is not biologically undesirable and includes that which is acceptable for veterinary use and/or human pharmaceutical use.

The term “pharmaceutical composition” is intended to encompass a drug product including the active ingredient(s), pharmaceutically acceptable excipients that make up the carrier, as well as any product which results, directly or indirectly, from combination, complexation or aggregation of any two or more of the ingredients. Accordingly, the pharmaceutical compositions of the present invention encompass any composition made by admixing the active ingredient, active ingredient dispersion or composite, additional active ingredient(s), and pharmaceutically acceptable excipients.

The expression “pharmaceutically acceptable salt” is meant those salts which are, within the scope of sound medical judgment, suitable for use in contact with the tissues of humans and lower animals without undue toxicity, irritation, allergic response and the like, commensurate with a reasonable benefit/risk ratio, and effective for their intended use. Representative alkali or alkaline earth metal salts include the sodium, calcium, potassium and magnesium salts, and the like.

The term “therapeutically effective amount” as used herein means the amount of a compound that, when administered to a mammal for treating a state, disorder or condition, is sufficient to effect such treatment. The “therapeutically effective amount” will vary depending on the compound, the disease and its severity and the age, weight, physical condition and responsiveness of the mammal to be treated.

The term “delivering” as used herein means providing a therapeutically effective amount of an active ingredient to a particular location within a host causing a therapeutically effective blood concentration of the active ingredient at the particular location. This can be accomplished, e.g., by topical, local or by systemic administration of the active ingredient to the host.

The term “buffering agent” as used herein is intended to mean a compound used to resist a change in pH upon dilution or addition of acid of alkali. Such compounds include, by way of example and without limitation, potassium metaphosphate, potassium phosphate, monobasic sodium acetate and sodium citrate anhydrous and dehydrate and other such material known to those of ordinary skill in the art.

The term “sweetening agent” as used herein is intended to mean a compound used to impart sweetness to a formulation. Such compounds include, by way of example and without limitation, aspartame, dextrose, glycerin, mannitol, saccharin sodium, sorbitol, sucrose, fructose and other such materials known to those of ordinary skill in the art.

The term “binders” as used herein is intended to mean substances used to cause adhesion of powder particles in granulations. Such compounds include, by way of example and without limitation, acacia alginic acid, tragacanth, carboxymethylcellulose sodium, polyvinylpyrrolidone, compressible sugar (e.g., NuTab), ethylcellulose, gelatin, liquid glucose, methylcellulose, povidone and pregelatinized starch, combinations thereof and other material known to those of ordinary skill in the art. If required, other binders may also be included in the present invention.

Exemplary binders include starch, polyethylene glycol, guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, polypropylene glycol, polyoxyethylene-polypropylene copolymer, polyethylene ester, polyethylene sorbitan ester, polyethylene oxide, microcrystalline cellulose, polyvinylpyrrolidone, combinations thereof and other such materials known to those of ordinary skill in the art.

The term “diluent” or “filler” as used herein is intended to mean inert substances used as fillers to create the desired bulk, flow properties, and compression characteristics in the preparation of solid dosage formulations. Such compounds include, by way of example and without limitation, dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art.

The term “glidant” as used herein is intended to mean agents used in solid dosage formulations to improve flow-properties during tablet compression and to produce an anti-caking effect. Such compounds include, by way of example and without limitation, colloidal silica, calcium silicate, magnesium silicate, silicon hydrogel, cornstarch, talc, combinations thereof and other such materials known to those of ordinary skill in the art.

The term “lubricant” as used herein is intended to mean substances used in solid dosage formulations to reduce friction during compression of the solid dosage. Such compounds include, by way of example and without limitation, calcium stearate, magnesium stearate, mineral oil, stearic acid, zinc stearate, combinations thereof and other such materials known to those of ordinary skill in the art.

The term “disintegrant” as used herein is intended to mean a compound used in solid dosage formulations to promote the disruption of the solid mass into smaller particles which are more readily dispersed or dissolved. Exemplary disintegrants include, by way of example and without limitation, starches such as corn starch, potato starch, pregelatinized, sweeteners, clays, such as bentonite, macrocrystalline cellulose (e.g. Avicel™), carsium (e.g. Amberlite™), alginates, sodium starch glycolate, gums such as agar, guar, locust bean, karaya, pectin, tragacanth, combinations thereof and other such materials known to those of ordinary skill in the art.

The term “wetting agent” as used herein is intended to mean a compound used to aid in attaining intimate contact between solid particles and liquids. Exemplary wetting agents include, by way of example and without limitation, gelatin, casein, lecithin (phosphatides), gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters, (e.g., TWEEN™s), polyethylene glycols, polyoxyethylene stearates colloidal silicon dioxide, phosphates, sodium dodecylsulfate, carboxymethylcellulose calcium, carboxymethylcellulose sodium, methylcellulose, hydroxyethylcellulose, hydroxylpropylcellulose, hydroxypropylmethylcellulose phthalate, noncrystalline cellulose, magnesium aluminum silicate, triethanolamine, polyvinyl alcohol, and polyvinylpyrrolidone (PVP). Tyloxapol (a nonionic liquid polymer of the alkyl aryl polyether alcohol type, also known as superinone or triton) is another useful wetting agent, combinations thereof and other such materials known to those of ordinary skill in the art.

As used herein, D_{X} means that X percent of the particles have a diameter less than a specified diameter D. Thus, a D₉₀ of less than 300 microns means that 90 volume-percent of the micronized particles in a composition have a diameter less than 300 microns.

The term “micronization” used herein means a process or method by which the size of a population of particles is reduced.

As used herein, the term “micron” or “μm” both are same refers to “micrometer” which is 1×10⁻⁶ meter.

As used herein, “crystalline particles” means any combination of single crystals, aggregates and agglomerates.

As used herein, “Particle Size Distribution (P.S.D)” means the cumulative volume size distribution of equivalent spherical diameters as determined by laser diffraction in Malvern Master Sizer 2000 equipment or its equivalent. “Mean particle size distribution, i.e., D₅₀” correspondingly, means the median of said particle size distribution.

By “substantially pure” is meant having purity greater than about 99%, specifically greater than about 99.5%, and more specifically greater than about 99.9% measured by HPLC.

### BRIEF DESCRIPTION OF THE DRAWINGS

FIG. 1 is a characteristic powder X-ray diffraction (XRD) pattern of amorphous darifenacin free base.

FIG. 2 is a characteristic infra red (IR) spectrum of amorphous darifenacin free base.

FIG. 3 is a characteristic powder X-ray diffraction (XRD) pattern of darifenacin hydrobromide polymorphic form A1.

FIG. 4 is a characteristic infra red (IR) spectrum of darifenacin hydrobromide polymorphic form A1.

FIG. 5 is a characteristic differential scanning calorimetric (DSC) thermogram of darifenacin hydrobromide polymorphic form A1.

FIG. 6 is a characteristic thermogravimetric analysis (TGA) of darifenacin hydrobromide polymorphic form A1.

The X-Ray powder diffraction was measured by an X-ray powder diffractometer equipped with a Cu-anode (?=1.54 Angstrom), X-ray source operated at 40 kV, 40 mA and a Ni filter is used to strip K-beta radiation. Two-theta calibration is performed using an NIST SRM 1976, Corundum standard. The sample was analyzed using the following instrument parameters: measuring range=3-45° 2?; step width=0.01579°; and measuring time per step=0.11 second.

DSC (Differential Scanning Calorimetry) measurements were performed with a Differential Scanning Calorimeter (DSC Q1000, TA Instruments, New Castle, Del., USA) at a scan rate of 10° C. per minute. The nitrogen gas purge is done at 50 mL/min. The instrument was calibrated for temperature and heat flow using indium as standards. The samples were encapsulated in to closed aluminium pans subsequently crimped to ensure a tight seal. Data acquisition and analysis were performed using Universal analysis 2000 software (TA Instruments).

Thergravimetric analysis was performed with a TGA Q500 of TA instruments, Lukens Drive, Delware, USA.

FT-IR spectroscopy was carried out with a Perkin Elmer Spectrum 100 series spectrometer. For the production of the KBr compacts approximately 2 mg of sample was powdered with 200 mg of KBr. The spectra were recorded in transmission mode ranging from 4000 or 3800 to 450 cm⁻¹.

### DETAILED DESCRIPTION OF THE INVENTION

According to another aspect of the present invention, there is provided a stable and substantially pure amorphous form of darifenacin free base.

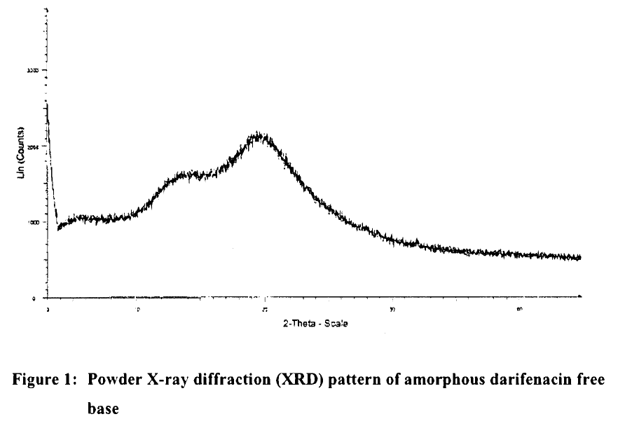

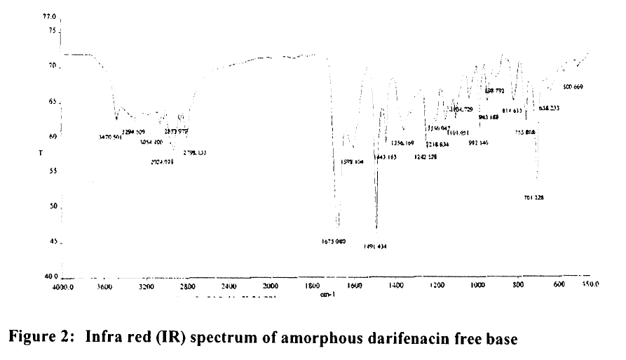

Amorphous form of darifenacin free base is characterized by at least one, and preferably all, of the following properties: a powder XRD pattern substantially in accordance with FIG. 1; an IR spectrum substantially in accordance with FIG. 2; and an IR spectrum having absorption bands at about 3470, 2924, 1675, 1598, 1491, 1443, 1356, 1242, 1218, 982, 943, 753 and 701 cm⁻¹. The X-ray powder diffraction pattern shows no peaks that are characteristic of amorphous form of darifenacin free base, thus demonstrating the amorphous nature of the product.

According to another aspect of the present invention, a process is provided for preparation of a stable and substantially pure amorphous form of darifenacin free base, which comprises:
a) providing a solution of darifenacin free base in a suitable solvent selected from the group comprising halogenated hydrocarbons, alcohols, ketones, and mixtures thereof;
b) optionally, filtering the solvent solution to remove any extraneous matter; and
c) substantially removing the solvent from the solution to afford amorphous form of darifenacin free base.

The process can produce amorphous darifenacin free base in substantially pure form.

The term “substantially pure amorphous form of darifenacin free base” refers to the amorphous form of darifenacin free base having purity greater than about 98%, specifically greater than about 99%, more specifically greater than about 99.5% and still more specifically greater than about 99.9% (measured by HPLC).

The amorphous darifenacin free base is stable, consistently reproducible and has good flow properties, and which is particularly suitable for bulk preparation and handling, and so, the novel amorphous darifenacin free base is suitable for formulating darifenacin. Moreover, the amorphous form of darifenacin free base is useful intermediate in the preparation of darifenacin pharmaceutically acceptable salts in high Purity.

Preferable halogenated hydrocarbons are dichloromethane, 1,2-dichloroethane, chloroform, carbon tetrachloride, and mixtures thereof, and more preferably dichloromethane. Exemplary alcohol solvents include, but are not limited to, C₁ to C₆ straight or branched chain alcohol solvents such as methanol, ethanol, isopropyl alcohol, butanol, amyl alcohol, hexanol, and mixtures thereof. Specific alcohol solvents are methanol, ethanol, isopropyl alcohol and mixtures thereof. Exemplary ketone solvents include, but are not limited to, acetone, methyl ethyl ketone, methyl isobutyl ketone, methyl tert-butyl ketone and the like, and mixtures thereof. Specific ketone solvent is acetone.

Step-(a) of providing a solution of darifenacin free base includes dissolving darifenacin free base in the solvent or obtaining an existing solution from a previous processing step.

Preferably the darifenacin free base is dissolved in the solvent at a temperature of below about boiling temperature of the solvent used, more preferably at about 25° C. to about 110° C., and still more preferably at about 30° C. to about 80° C.

The solution in step-(a) may also be prepared by reacting 3(S)-(−)-(1-carbamoyl-1,1-diphenylmethyl)-1-pyrrolidine or an acid addition salt thereof with 5-(2-bromoethyl)-2,3-dihydrobenzofuran in the presence of a suitable base, in a suitable solvent under suitable conditions to produce a reaction mass containing crude darifenacin free base followed by usual work up such as washings, extractions etc., and dissolving the resulting crude darifenacin free base in a solvent selected from the group comprising halogenated hydrocarbons, alcohols, ketones, and mixtures thereof, at a temperature of below about boiling temperature of the solvent used, more preferably at about 25° C. to about 110° C. and still more preferably at about 25° C. to about 80° C.

Alternatively, the solution in step-(a) may be prepared by treating an acid addition salt of darifenacin with a base to liberate darifenacin free base and dissolving or extracting the darifenacin free base in a solvent selected from the group comprising halogenated hydrocarbons, alcohols, ketones, and mixtures thereof.

As acid addition salts, the salts derived from a therapeutically acceptable acid such as hydrochloric acid, hydrobromic acid, sulfuric acid, nitric acid, acetic acid, propionic acid and, phosphoric acid, succinic acid, maleic acid, fumaric acid, citric acid, glutaric acid, citraconic acid, glutaconic acid, tartaric acid, malic acid, and ascorbic acid can be used. More preferable salt is being darifenacin hydrobromide.

The treatment of an acid addition salt with base is carried out in any solvent and the selection of solvent is not critical. A wide variety of solvents such as chlorinated solvents, hydrocarbon solvents, ether solvents etc., can be used.

The base can be inorganic or organic. Preferable base is an inorganic base selected from alkali metal hydroxides, carbonates and bicarbonates. Preferable alkali metal is sodium or potassium.

The solution obtained in step-(a) may optionally be subjected to carbon treatment. The carbon treatment can be carried out by methods known in the art, for example by stirring the solution with finely powdered carbon at a temperature of below about 70° C. for at least 15 minutes, preferably at a temperature of about 40° C. to about 70° C. for at least 30 minutes; and filtering the resulting mixture through hyflo to obtain a filtrate containing darifenacin by removing charcoal. Preferably, finely powdered carbon is an active carbon.

The solution obtained in step-(a) or step-(b) is optionally stirred at a temperature of about 30° C. to the reflux temperature of the solvent used for at least 20 minutes, and preferably at a temperature of about 40° C. to the reflux temperature of the solvent used from about 30 minutes to about 4 hours.

Removal of solvent in step-(c) is accomplished by, for example, substantially complete evaporation of the solvent, concentrating the solution and filtering the solid under inert atmosphere. Alternatively, the solvent may also be removed by evaporation. Evaporation can be achieved at sub-zero temperatures by the lyophilization or freeze-drying technique. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying to obtain a dry amorphous powder.

One of the preferred methodologies to remove the solvent involves spray-drying, in which a solution of darifenacin is sprayed into the spray drier at the flow rate ranging from 10 to 300 ml/hr, preferably flow rate is 40 to 200 ml/hr. The air inlet temperature to the spray drier used may range from 25 to 150° C., preferably from 60° C. to 110° C. and the outlet air temperature used may range from 30 to 90° C. Another preferred method is vertical agitated thin-film drying (or evaporation). Agitated thin film evaporation technology involves separating the volatile component using indirect heat transfer coupled with mechanical agitation of the flowing film under controlled condition.

The distillation process can be performed at atmospheric pressure or reduced pressure. Preferably the solvent is removed at a pressure of about 760 mm Hg or less, more preferably at about 400 mm Hg or less, still more preferably at about 80 mm Hg or less, and most preferably from about 30 to about 80 mm Hg. The substantially pure amorphous form of darifenacin obtained by the above processes may be further dried in, for example, Vacuum Tray Dryer, Rotocon Vacuum Dryer, Vacuum Paddle Dryer or pilot plant Rota vapor, to further lower residual solvents.

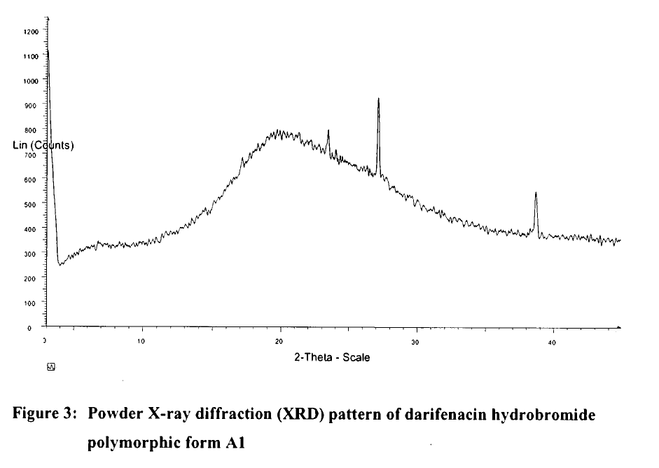

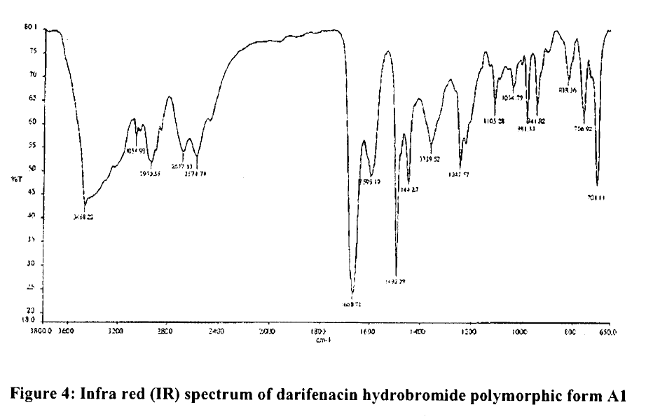

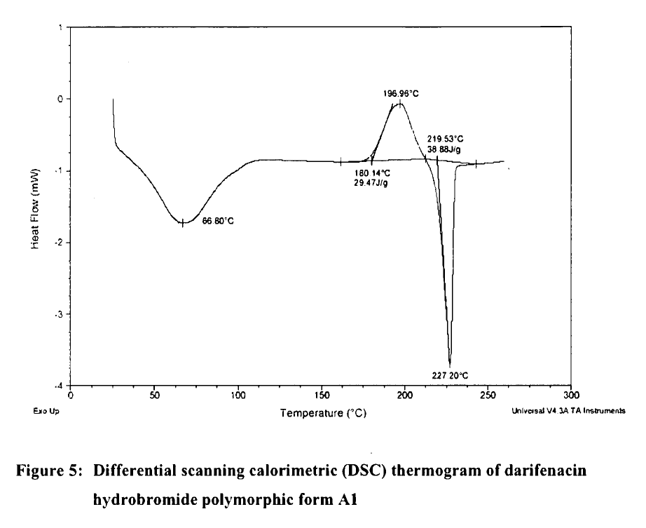

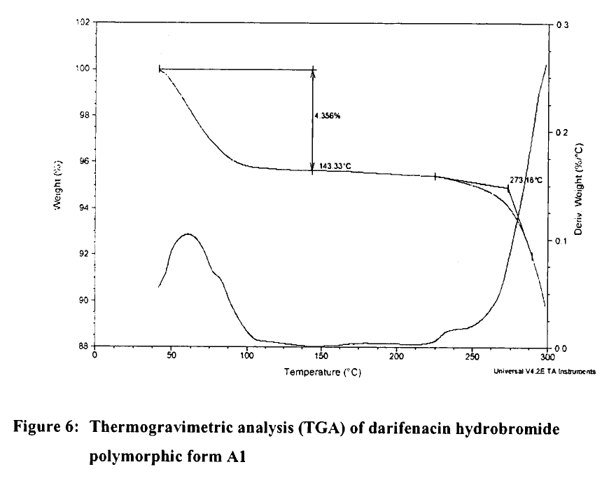

According to another aspect of the present invention, there is provided a novel polymorphic form of darifenacin hydrobromide, designated as polymorphic form A1, characterized by at least one, and preferably all, of the following properties:
i) a powder X-ray diffraction pattern substantially in accordance with FIG. 3;
ii) a powder X-ray diffraction pattern having peaks at about 23.44, 27.12 and 36.68±0.2 degrees 2-theta substantially as depicted in FIG. 3;
iii) an IR spectrum substantially in accordance with FIG. 4;
iv) an IR spectrum having absorption bands at about 3468, 2930, 1668, 1595, 1492, 1444, 1359, 1242, 1220, 981, 942, 757 and 704 cm⁻¹;
v) a DSC thermogram substantially in accordance with FIG. 5; and
vi) a TGA thermogram substantially in accordance with FIG. 6.

According to another aspect of the present invention, a process is provided for preparation of a stable and substantially pure darifenacin hydrobromide polymorphic form A1, which comprises:
a) providing a solution of darifenacin hydrobromide in a solvent selected from the group comprising water, methanol, ethanol, n-propanol, isopropyl alcohol, dichloromethane, acetonitrile, and mixtures thereof;
b) optionally, filtering the solvent solution to remove any extraneous matter; and
c) substantially removing the solvent from the solution to afford polymorphic form A1 of darifenacin hydrobromide.

The process can produce polymorphic form A1 of darifenacin hydrobromide in substantially pure form.

The term “substantially pure darifenacin hydrobromide polymorphic form A1” refers to the darifenacin hydrobromide polymorphic form A1 having purity greater than about 98%, specifically greater than about 99%, more specifically greater than about 99.5% and still more specifically greater than about 99.9% (measured by HPLC).

The darifenacin hydrobromide polymorphic form A1 is stable, consistently reproducible and has good flow properties, and which is particularly suitable for bulk preparation and handling, and so, the novel darifenacin hydrobromide polymorphic form A1 is suitable for formulating darifenacin.

Step-(a) of providing a solution of darifenacin hydrobromide includes dissolving darifenacin hydrobromide in the solvent, or obtaining an existing solution from a previous processing step.

Preferably the darifenacin hydrobromide is dissolved in the solvent at a temperature of below about boiling temperature of the solvent used, more preferably at about 25° C. to about 100° C., and still more preferably at about 30° C. to about 80° C.

The solution in step-(a) may also be prepared by reacting 3(S)-(−)-(1-carbamoyl-1,1-diphenylmethyl)-1-pyrrolidine or an acid addition salt thereof with 5-(2-bromoethyl)-2,3-dihydrobenzofuran in the presence of a suitable base, in a suitable solvent under suitable conditions to produce a reaction mass containing crude darifenacin free base followed by treatment with aqueous hydrobromic acid to produce a solution containing darifenacin hydrobromide, or optionally subjecting the solution to usual work up such as washings, extractions etc., and dissolving the resulting darifenacin hydrobromide in a suitable solvent at a temperature of below about boiling temperature of the solvent used, more preferably at about 25° C. to about 110° C. and still more preferably at about 25° C. to about 80° C.

The solution obtained in step-(a) may optionally be subjected to carbon treatment. The carbon treatment can be carried out by methods known in the art, for example by stirring the solution with finely powdered carbon at a temperature of below about 70° C. for at least 15 minutes, preferably at a temperature of about 40° C. to about 70° C. for at least 30 minutes; and filtering the resulting mixture through hyflo to obtain a filtrate containing darifenacin hydrobromide by removing charcoal. Preferably, finely powdered carbon is an active carbon.

The solution obtained in step-(a) or step-(b) is optionally stirred at a temperature of about 30° C. to the reflux temperature of the solvent used for at least 20 minutes, and preferably at a temperature of about 40° C. to the reflux temperature of the solvent used from about 30 minutes to about 4 hours.

Removal of solvent in step-(c) is accomplished by, for example, substantially complete evaporation of the solvent, concentrating the solution and filtering the solid under inert atmosphere. Alternatively, the solvent may also be removed by evaporation. Evaporation can be achieved at sub-zero temperatures by the lyophilisation or freeze-drying technique. The solution may also be completely evaporated in, for example, a pilot plant Rota vapor, a Vacuum Paddle Dryer or in a conventional reactor under vacuum above about 720 mm Hg by flash evaporation techniques by using an agitated thin film dryer (“ATFD”), or evaporated by spray drying.

The distillation process can be performed at atmospheric pressure or reduced pressure. Preferably the solvent is removed at a pressure of about 760 mm Hg or less, more preferably at about 400 mm Hg or less, still more preferably at about 80 mm Hg or less, and most preferably from about 30 to about 80 mm Hg.

The substantially pure darifenacin hydrobromide polymorphic form A1 obtained by the above processes may be further dried in, for example, Vacuum Tray Dryer, Rotocon Vacuum Dryer, Vacuum Paddle Dryer or pilot plant Rota vapor, to further lower residual solvents.

In one embodiment, the substantially pure darifenacin hydrobromide polymorphic form A1 disclosed herein for use in the pharmaceutical compositions of the present invention, wherein 90 volume-percent of the particles (D₉₀) have a size of less than or equal to about 400 microns, specifically less than or equal to about 300 microns, more specifically less than or equal to about 200 microns, still more specifically less than or equal to about 100 microns, and most specifically less than or equal to about 15 microns.

In another embodiment, the particle sizes of substantially pure darifenacin hydrobromide polymorphic form A1 can be achieved by a mechanical process of reducing the size of particles which includes any one or more of cutting, chipping, crushing, milling, grinding, micronizing, trituration or other particle size reduction methods known in the art, to bring the solid state forms the desired particle size range.

According to another aspect of the present invention, there is provided a method for treating or preventing overactive bladder with symptoms of urge urinary incontinence, comprising administering the darifenacin hydrobromide polymorphic form A1, or a pharmaceutical composition that comprises darifenacin hydrobromide polymorphic form A1, along with pharmaceutically acceptable excipients.

According to another aspect of the present invention, there is provided pharmaceutical compositions comprising amorphous darifenacin free base and one or more pharmaceutically acceptable excipients.

According to another aspect of the present invention, there is provided pharmaceutical compositions comprising amorphous darifenacin free base prepared according to processes of the present invention in any of its embodiments and one or more pharmaceutically acceptable excipients.

According to another aspect of the present invention, there is provided a process for preparing a pharmaceutical formulation comprising combining amorphous darifenacin free base prepared according to processes of the present invention in any of its embodiments, with one or more pharmaceutically acceptable excipients.

According to another aspect of the present invention, there is provided pharmaceutical compositions comprising darifenacin hydrobromide polymorphic form A1 and one or more pharmaceutically acceptable excipients.

According to another aspect of the present invention, there is provided pharmaceutical compositions comprising darifenacin hydrobromide polymorphic form A1 prepared according to processes of the present invention in any of its embodiments and one or more pharmaceutically acceptable excipients.

According to another aspect of the present invention, there is provided a process for preparing a pharmaceutical formulation comprising combining darifenacin hydrobromide polymorphic form A1 prepared according to processes of the present invention in any of its embodiments, with one or more pharmaceutically acceptable excipients.

Yet another embodiment of the present invention is directed to pharmaceutical compositions comprising at least a therapeutically effective amount of substantially pure polymorphic forms of darifenacin free base and its hydrobromide salt of the present invention. Such pharmaceutical compositions may be administered to a mammalian patient in any dosage form, e.g., liquid, powder, elixir, injectable solution, etc. Dosage forms may be adapted for administration to the patient by oral, buccal, parenteral, ophthalmic, rectal and transdermal routes or any other acceptable route of administration. Oral dosage forms include, but are not limited to, tablets, pills, capsules, troches, sachets, suspensions, powders, lozenges, elixirs and the like. The polymorphic forms of darifenacin free base and its hydrobromide salt of the present invention may also be administered as suppositories, ophthalmic ointments and suspensions, and parenteral suspensions, which are administered by other routes. The dosage forms may contain any one of the polymorphic forms of darifenacin free base and its hydrobromide salt of the present invention as is or, alternatively, may contain any one of the polymorphic forms of darifenacin free base and its hydrobromide salt of the present invention as part of a composition. The pharmaceutical compositions may further contain one or more pharmaceutically acceptable excipients. Suitable excipients and the amounts to use may be readily determined by the formulation scientist based upon experience and consideration of standard procedures and reference works in the field, e.g., the buffering agents, sweetening agents, binders, diluents, fillers, lubricants, wetting agents and disintegrants described hereinabove.

Capsule dosages will contain any one of the polymorphic forms of darifenacin free base and its hydrobromide salt of the present invention within a capsule which may be coated with gelatin. Tablets and powders may also be coated with an enteric coating. The enteric-coated powder forms may have coatings containing at least phthalic acid cellulose acetate, hydroxypropylmethyl cellulose phthalate, polyvinyl alcohol phthalate, carboxy methyl ethyl cellulose, a copolymer of styrene and maleic acid, a copolymer of methacrylic acid and methyl methacrylate, and like materials, and if desired, they may be employed with suitable plasticizers and/or extending agents. A coated capsule or tablet may have a coating on the surface thereof or may be a capsule or tablet comprising a powder or granules with an enteric-coating.

Tableting compositions may have few or many components depending upon the tableting method used, the release rate desired and other factors. For example, the compositions of the present invention may contain diluents such as cellulose-derived materials like powdered cellulose, microcrystalline cellulose, microtine cellulose, methyl cellulose, ethyl cellulose, hydroxyethyl cellulose, hydroxypropyl cellulose, hydroxypropylmethyl cellulose, carboxymethyl cellulose salts and other substituted and unsubstituted celluloses; starch; pregelatinized starch; inorganic diluents such calcium carbonate and calcium diphosphate and other diluents known to one of ordinary skill in the art. Yet other suitable diluents include waxes, sugars (e.g. lactose) and sugar alcohols like mannitol and sorbitol, acrylate polymers and copolymers, as well as pectin, dextrin and gelatin.

Other excipients contemplated by the present invention include binders, such as acacia gum, pregelatinized starch, sodium alginate, glucose and other binders used in wet and dry granulation and direct compression tableting processes; disintegrants such as sodium starch glycolate, crospovidone, low-substituted hydroxypropyl cellulose and others; lubricants like magnesium and calcium stearate and sodium stearyl fumarate; flavorings; sweeteners; preservatives; pharmaceutically acceptable dyes and glidants such as silicon dioxide.

The following examples are provided to enable one skilled in the art to practice the invention and are merely illustrate the process of this invention. However, it is not intended in any way to limit the scope of the present invention.

### Reference Example

### Preparation of Darifenacin Hydrobromide

The reaction mixture of 3(S)-(−)-(1-cabamoyl-1,1-diphenylmethyl)-1-pyrrolidine (32.5 gm), potassium carbonate (45.26 gm) and 5-(2-bromoethyl)-2,3-dihydrobenzofuran (31.65 gm) in acetonitrile (450 ml) was heated at 70-75° C. and maintained for 2 hours. The insoluble material was filtered and washed with acetonitrile (50 ml). Acetonitrile was distilled out completely followed by the addition of acetone (500 ml) and aqueous HBr (17.5 gm) at 10-15° C. The reaction mixture was stirred for 6 hours at 20-25° C. followed by cooling at 0° C. and stirred for 1 hour at 0-5° C. The solid mass was filtered and washed with acetone (50 ml). The material was dried at 50-55° C. to produce 42 gm of darifenacin hydrobromide.

### EXAMPLES

### Example 1

### Preparation of Amorphous Darifenacin Base

Darifenacin Hydrobromide (10 gm) was taken in water (500 ml) at 20-25° C. and the pH was adjusted to 12±0.5 with 20% NaOH solution. The product was extracted with dichloromethane (500 ml) and the organic layer was washed with water (100 ml). The resulting methylene chroride layer was distilled under vacuum to produce amorphous darifenacin base (Yield: 7.5 gm).

### Example 2

### Preparation of Polymorphic Form A1 of Darifenacin Hydrobromide

A mixture of 3(S)-(−)-(1-cabamoyl-1,1-diphenylmethyl)-1-pyrrolidine-L(+)-tartrate (10 gm), potassium carbonate (16 gm), 5-(2-bromoethyl)-2,3-dihydrobenzofuran (6.3 gm) and acetonitrile (450 ml) were refluxed at 70-75° C. for 2 hours and then cooled at 20-25° C. The reaction mass was filtered to remove the salt and washed with acetonitrile (50 ml). The aqueous hydrobromic acid (4 g) was added to the filtrate at 10-15° C. This was followed by the distillation of filtrate under vacuum at below 40° C. The resulted mass was dried to produce 7.0 gm of darifenacin hydrobromide polymorphic form A1.

### Example 3

### Preparation of Polymorphic Form A1 of Darifenacin Hydrobromide

Darifenacin hydrobromide (5 gm, obtained in reference example 1) was suspended in dichloromethane (70 ml) at 25-30° C. The suspension was stirred for 15 minutes to obtain a clear solution. The resulted solution was filtered and filtrate was distilled out completely under vacuum and dried to produce 4.8 gm of darifenacin hydrobromide polymorphic form A1.

### Example 4

### Preparation of Polymorphic Form A1 of Darifenacin Hydrobromide

Darifenacin hydrobromide (5 gm) was dissolved in Methanol (100 ml) at 25-30° C. The solution was passed through spray drier, at the rate of 200 ml per hour at 95° C. of the inlet temperature. The resulted material dried to give 3.6 gm of darifenacin hydrobromide polymorphic form A1.

1. A polymorphic form A1 of darifenacin hydrobromide characterized by at least one of the following properties:
i) a powder X-ray diffraction pattern substantially in accordance with FIG. 3;
ii) a powder X-ray diffraction pattern having peaks at about 23.44, 27.12 and 36.68±0.2 degrees 2-theta;
iii) an IR spectrum substantially in accordance with FIG. 4;
iv) an IR spectrum having absorption bands at about 3468, 2930, 1668, 1595, 1492, 1444, 1359, 1242, 1220, 981, 942, 757 and 704 cm⁻¹;
v) a DSC thermogram substantially in accordance with FIG. 5; and
vi) a TGA thermogram substantially in accordance with FIG. 6.

2. A process for the preparation of darifenacin hydrobromide polymorphic form A1 of claim 1, which comprises:
a) providing a solution of darifenacin hydrobromide in a solvent selected from the group consisting of water, methanol, ethanol, n-propanol, isopropyl alcohol, dichloromethane, acetonitrile, and mixtures thereof;
b) optionally, filtering the solvent solution to remove any extraneous matter; and
c) substantially removing the solvent from the solution to afford polymorphic form A1 of darifenacin hydrobromide.

3. The process of claim 2, wherein the solution in step-(a) is prepared by dissolving darifenacin hydrobromide in the solvent at a temperature of below about boiling temperature of the solvent used; and wherein the solution obtained in step-(a) is optionally subjected to carbon treatment.

4. The process of claim 3, wherein the dissolution is carried out at a temperature of about 25° C. to about 100° C.

5. (canceled)

6. The process of claim 2, wherein the solution obtained in step-(a) or step-(b) is optionally stirred at a temperature of about 30° C. to the reflux temperature of the solvent used for at least 20 minutes; and wherein the removal of the solvent in step-(c) is accomplished by complete evaporation of the solvent, spray drying, vacuum drying, lyophilization or freeze drying, or a combination thereof.

7. (canceled)

8. Amorphous form of darifenacin free base characterized by at least one of the following properties:
i) a powder XRD pattern substantially in accordance with FIG. 1; and/or
ii) an IR spectrum substantially in accordance with FIG. 2; and
iii) an IR spectrum having absorption bands at about 3470, 2924, 1675, 1598, 1491, 1443, 1356, 1242, 1218, 982, 943, 753 and 701 cm⁻¹.

9. A process for the preparation of amorphous darifenacin free base of claim 8, which comprises:
a) providing a solution of darifenacin free base in a solvent selected from group consisting of dichloromethane, 1,2-dichloroethane, chloroform, carbon tetrachloride, methanol, ethanol, isopropyl alcohol, butanol, amyl alcohol, hexanol, acetone, methyl ethyl ketone, methyl isobutyl ketone, methyl tert-butyl ketone, and mixtures thereof.
b) optionally, filtering the solvent solution to remove any extraneous matter; and
c) substantially removing the solvent from the solution to afford amorphous form of darifenacin free base.

10. (canceled)

11. The process of claim 9, wherein the solvent is selected from group consisting of dichloromethane, methanol, acetone, and mixtures thereof.

12. The process of claim 9, wherein the solution in step-(a) is prepared either by dissolving darifenacin free base in the solvent at a temperature of below about boiling temperature of the solvent used, or by treating an acid addition salt of darifenacin with a base to liberate darifenacin free base and dissolving or extracting the darifenacin free base in the solvent at a temperature of below about boiling temperature of the solvent used.

13. (canceled)

14. (canceled)

15. (canceled)

16. The process of claim 12, wherein the acid addition salt is derived from a therapeutically acceptable acid selected from the group consisting of hydrochloric acid, hydrobromic acid, sulfuric acid, nitric acid, acetic acid, propionic acid, phosphoric acid, succinic acid, maleic acid, fumaric acid, citric acid, glutaric acid, citraconic acid, glutaconic acid, tartaric acid, malic acid, and ascorbic acid; and wherein the base is an inorganic or organic base.

17. The process of claim 16, wherein the acid is hydrobromic acid; and wherein the inorganic base is selected from alkali metal hydroxides, carbonates and bicarbonates.

18. (canceled)

19. (canceled)

20. (canceled)

21. The process of claim 9, wherein the solution obtained in step-(a) or step-(b) is optionally stirred at a temperature of about 30° C. to the reflux temperature of the solvent used for at least 20 minutes; and wherein the removal of the solvent in step-(c) is accomplished by complete evaporation of the solvent, spray drying, vacuum drying, lyophilization or freeze drying, or a combination thereof.

22. (canceled)

23. A pharmaceutical composition comprising amorphous darifenacin free base of claim 8 and one or more pharmaceutically acceptable excipients.

24. (canceled)

25. A pharmaceutical composition comprising darifenacin hydrobromide polymorphic form A1 of claim 1 and one or more pharmaceutically acceptable excipients.

26. A process for preparing the pharmaceutical composition of claim 25, comprising combining darifenacin hydrobromide polymorphic form A1 with one or more pharmaceutically acceptable excipients.

27. The pharmaceutical composition of anyone of claims 23 and 25, wherein the pharmaceutical composition is selected from dosage forms comprising liquid, powder, elixir and injectable solution.

28. The pharmaceutical composition of claim 27, wherein the pharmaceutical composition is selected from a solid dosage form and an oral suspension.

29. A pharmaceutical composition comprising crystalline particles of pure darifenacin hydrobromide polymorphic form A1, wherein 90 volume-% of the particles (D₉₀) have a size of less than or equal to about 400 microns; less than or equal to about 300 microns; less than or equal to about 100 microns; or less than or equal to about 15 microns.

30. (canceled)

31. (canceled)

32. (canceled)
